(19) United States
(12) Patent Application Publication
(10) Pub. No.: US 2007/0123782 A1
(43) Pub. Date: May 31, 2007

(54) On-site healthcare diagnostic device

(76) Inventors: Jackson Connolly, Post Falls, ID (US); Christopher T. Maus, Sagle, ID (US)

Correspondence Address: MEHRMAN LAW OFFICE, P.C., ONE PREMIER PLAZA, 5605 GLENRIDGE DRIVE, STE. 795, ATLANTA, GA, 30342, US

(21) Appl. No.: 11/605,490

(22) Filed: Nov. 28, 2006

Related U.S. Application Data
(60) Provisional application No. 60/740,966, filed on Nov. 30, 2005.

(51) Int. Cl. A61B 5/02; C12Q 1/60; C12Q 1/54

(52) U.S. Cl. 600/483; 600/485; 600/490; 435/14; 435/11

(57) ABSTRACT

A on-site healthcare diagnostic device containing a blood pressure monitor and one or more test strip readers, such as a blood sugar or cholesterol test strip reader. The on-site device may be deployed as a special purpose unit or through accessories used in connection with a general purpose computer. In the special purpose unit, the on-site device includes a pump and control electronics for the blood pressure monitor and at least one test strip reader carried by an integral housing that also carries a display device and user interface, such as a three-button keypad, for operating the device. The on-site device may be expanded to accommodate external test strip readers and other diagnostic devices connected through one or more universal data ports.

### CROSS REFERENCE TO RELATED APPLICATIONS

This patent application claims priority to commonly owned U.S. Provisional Patent Application No. 60/740,966 entitled “Health Risk Assessment Devices” filed Nov. 30, 2005, which is incorporated herein by reference. This patent application also incorporates by reference the disclosures of U.S. Pat. No. 6,602,469, U.S. Pat. No. 7,092,891 and U.S. patent application Publication No. 2002/0128864.

### TECHNICAL FIELD

This patent application relates to the field of healthcare diagnostic devices and, more particularly, to an on-site healthcare diagnostic devices, that is diagnostic devices that may be deployed in a physician's office, home, or workplace.

### BACKGROUND

Great advances have been made in understanding the basis for human disease such as heart disease, diabetes, liver disease etc. Significant improvements in healthcare diagnostic devices, such as blood pressure monitors, blood sugar monitors, blood cholesterol monitors, and so forth have also been made. Frequently diagnostic testing helps patients maintain greater awareness the wellness indicators measured by these devices, which improves their cognizance of these factors and improves compliance with therapy and preventative measures. Yet, diagnostic testing still proceeds largely in the same manner as it has for the last fifty years: a patient visits his or her physician's office, a nurse or other trained person takes fluid samples, the samples are sent to a laboratory, and in a few days a report is received by the physician's office, and the physician reviews it, makes a diagnosis and informs the patient. If the results show a problem, the patient must return for further testing.

A solution might be to have the patient or physician's office purchase a large number of diagnostic devices and or set up an on-site laboratory. This can be prohibitively expensive. In addition, learning how to use and keeping track of a large number of healthcare diagnostic devices can be inconvenient and time consuming. In general, there is an on-going need to make healthcare diagnostic devices less expensive and complex and thereby make important, potential lifesaving knowledge and technologies available to a wider population.

Therefore, even as the diagnostic knowledge and technology continues to improve and come down in price, many people may not take full advantage of the available technology and using the knowledge and technology can take days. Accordingly, there is an on-going need for on-site healthcare diagnostic devices that are less expensive and more convenient to use.

### SUMMARY OF THE INVENTION

The present invention meets the needs described above in a healthcare diagnostic device that permits one or more diagnostic tests that traditionally have required a laboratory and significant time to perform to be performed in a physican's office. The invention also provides a healthcare diagnostic device the permits diagnostic test that have traditionally been performed in a physician's office, with or without the cooperation of a laboratory, to be performed at the patient's home or workplace. A preferred embodiment includes a blood pressure monitor and one or more test strip readers, such as a blood cholesterol test strip reader. Another preferred embodiment includes a diagnostic device, such as a test strip reader, with a personal computer.

The on-site healthcare diagnostic device may also be embodied in a special purpose device that includes a pump and data interface for a blood pressure cuff, a test strip reader, a display, a user interface, and a controller for operating these devices. In particular, the display may be a small liquid crystal display (LCD) and the use interface may be a small three-button keypad. The device may also include a calibration interface for receiving and reading a removable calibration device that contains calibration data for test strips to be read by the test strip reader. For example, the calibration interface may be a romkey socket configured to receive a romkey that comes packaged with an associated set of test strips. The device may also include an interface for receiving and exchanging data with a removable memory storage device, such as a smartcard. Alternatively or additionally, the device may include a data port, such as a USB port, and associated data cable.

The on-site healthcare diagnostic device may further include multiple test strip readers and associated calibration interfaces in a special purpose device. For example, the device may include a first test strip reader for total cholesterol test strips and a second test strip reader for blood sugar test strips. In another embodiment, the device may include a first test strip reader for total cholesterol test strips, a second test strip reader for HDL cholesterol test strips, and a third test strip reader for triglyceride test strips. In this case, the device may be configured to compute LD cholesterol from readings receive from these readers and produce a full lipid panel measurement. The device may also include a data port, such as a USB data port, for connecting to an external test strip reader so that different types of test strip readers can be connected to the device, as desired.

In another embodiment, the on-site healthcare diagnostic device may implemented through a general purpose computer configured to interface with one or more external healthcare diagnostic devices, such as a blood pressure monitor and/or one or more test strip readers. In particular, the external healthcare diagnostic device may be configured to use a conventional data port, such as a USB port. In this case, the display, central processing unit and user interface of the general purpose computer can be used to operate the external healthcare diagnostic device, which removes components, complexity and cost from the individual healthcare diagnostic devices. This configuration also allows the general purpose computer to run application software and engage in network-based support for the healthcare diagnostic device, which produces a powerful and flexible paradigm for healthcare diagnostic devices and supporting systems.

For any of these embodiments, the on-site healthcare diagnostic device may be configured to work cooperatively with application software for calibrating or maintaining the diagnostic device and for calibrating, storing, analyzing and maintaining the medical data created by the diagnostic devices. In particular, the medical data may be downloaded onto a secure medical records maintenance facility for permanent storage and access by qualified medical professionals. The on-site healthcare diagnostic device may also for a part of a comprehensive wellness monitoring system that allows the user to track a wide range of medical, diet, exercise and other parameters in the convenience of their own home or workplace

The invention provides an on-site healthcare diagnostic device, comprising: a general purpose computer comprising a data port; one or more test strip readers connectable to the general purpose computer through the data port; and software running on the general purpose computer operable for driving the one or more test strip readers connected to the universal data port to operate in conjunction with a display, a user interface and a controller resident as part of the general purpose computer to function an as a healthcare diagnostic device. Preferably, the computer further includes a memory containing calibration data for the one or more test strip readers. Preferably, the computer includes a network interface for connecting to an off-site computer. Preferably, the on-site healthcare diagnostic device further includes an off-site computer connected to the network interface, the off-site computer including a memory containing calibration data for the one or more test strip readers. Preferably, the one or more test strip readers are capable of reading a test strip for measuring an analyte selected from the group consisting of LDL cholesterol, HDL cholesterol, total cholesterol, triglycerides, glucose and creatinine

The invention also provides a method of measuring the concentration of an analyte in a whole blood sample, the method comprising: connecting a dry test strip reader to a personal computer; applying the whole blood sample to the dry test strip; inserting the dry test strip into the test strip reader; reading the dry test strip and producing data representative of the concentration of the analyte; communicating the data to the personal computer; and displaying on the personal computer the concentration of the analyte. Preferably, the method further comprises using calibration data stored on the personal computer to determine the concentration of the analyte. Preferably, the method further comprises sending the calibration data to the personal computer via a network. Preferably, the analyte is selected from the group consisting of LDL cholesterol, HDL cholesterol, total cholesterol, triglycerides, glucose and creatinine.

In view of foregoing, it will be appreciated that the present invention provides a on-site healthcare diagnostic device that includes a blood pressure monitor and a test strip reader, such as a blood sugar or cholesterol testing device suitable for home-based used, that improves over conventional single-test diagnostic devices. The on-site device is also an flexible and expandable through the use of external diagnostic devices, and may be implemented using a special-purpose monitor or a general purpose computer. Specific structures for implementing the invention, and achieving the advantages of the invention described above, will be further understood with reference to the following detailed description and the appended drawings and claims. Although the following specific structures may be used to implement the invention, the invention is not limited to these specific embodiments, but is instead defined broadly in accordance with the claims at the end of this specification.

### BRIEF DESCRIPTION OF THE FIGURES

FIG. 1 is a function block diagram of a on-site health diagnostic device and related systems.

FIG. 2 is a perspective view of a on-site health diagnostic device including a blood pressure monitor and test strip reader, such as a blood cholesterol or blood sugar test strip reader.

FIG. 3 is a partially exploded view of the on-site health diagnostic device of FIG. 2 showing removable components in removed positions.

FIG. 4 is a perspective view of a on-site health diagnostic device with a USB port and a USB cable accessory.

FIG. 5 is a front view of a on-site health diagnostic device with a USB port and a USB memory stick accessory.

FIG. 6 is a perspective rear view of a on-site health diagnostic device that includes a USB port and a USB cable.

FIG. 7 is a rear view of the on-site health diagnostic device of FIG. 6.

FIG. 8 is a front view of an external test strip reader for use with a on-site health diagnostic device.

FIG. 9 is a block diagram of a on-site health diagnostic device with a blood pressure cuff interface and multiple test strip readers.

FIG. 10 is a block diagram of a on-site health diagnostic device implemented with a personal computer and an external test strip reader connected to the computer through a USB port.

FIG. 11 is a block diagram of a on-site health diagnostic device implemented with a personal computer, an external test strip reader, and a network interface.

FIG. 12 is a block diagram of an expandable on-site health diagnostic device implemented with a personal computer, multiple external test strip readers, and a network interface.

### DETAILED DESCRIPTION OF THE EMBODIMENTS

The present invention may be embodied as a on-site healthcare diagnostic device (“on-site device”) containing a blood pressure monitor and one or more test strip readers. Here, “on-site” means that the device can be used to perform diagnostic functions in the physician's office that traditionally have required the cooperation of a laboratory, or that the device can be used to perform diagnostic functions in the home or workplace that traditionally have been performed in a physican's office with or without the cooperation of a laboratory. The on-site device may be deployed as a special purpose unit or through accessories used in connection with a general purpose computer. In the special purpose unit, the on-site device preferably includes a pump and control electronics for the blood pressure monitor and at least one test strip reader carried by an integral housing that preferably also carries a display device and user interface, such as a three-button keypad, for operating the device. In the case of the accessorized general purpose computer, the pump for the blood pressure monitor and test strip reader are selectively connected to the host computer through one or more universal data ports, such as USB ports. In either case, the on-site device may be expanded to accommodate additional test strip readers and potentially other types of diagnostic or other devices through universal data ports.

In particular, the on-site device may include a blood pressure monitor and a total blood cholesterol monitor utilizing an optical test strip reader. Alternatively, the on-site device may include a blood pressure monitor and a blood sugar monitor utilizing an optical test strip reader, a biosensor test strip reader, or any other suitable type test strip reader. In fact, because the on-site device is expandable through the use of external diagnostic devices connected through a universal data port, it may include any number of test strip readers or other types of external diagnostic devices. For example, the on-site device, as expanded through the use of external devices, may a blood pressure monitor in on-site with a blood sugar monitor using a first test strip reader and a cholesterol monitor using a second test strip reader. The on-site device may further include a total cholesterol monitor, an HDL cholesterol monitor, and a triglyceride monitor. This combination will permit the on-site device to compute a LDL blood cholesterol value from the other measurements and produce a full lipid panel measurement. The device may also include a glucose monitor and a creatinine monitor. Alternatively, LDL may be measured directly as disclosed in U.S. patent application Ser. No. 11/206,893 filed Aug. 17, 2005, which is hereby incorporated by reference to the same extent as though fully disclosed herein. Or it may include a single monitor capable of reading a test strip for measuring one or more analytes selected from the group consisting of LDL cholesterol, HDL cholesterol, total cholesterol, triglycerides, glucose and creatinine. Details of a test strip and monitor are disclosed in U.S. patent application Ser. No. 11/207,121 filed Aug. 17, 2005, which is hereby incorporated by reference to the same extent as though fully disclosed herein. Additional diagnostic devices may also be used, such as a keytone test strip reader and any other type of test strip reader or other diagnostic device available with current technology or developed in the future.

The on-site device may include a number of different types of data storage and communication devices, such as a smartcard drive, a network interface, and one or more USB ports. Through the network interface, the on-site device may access a network-based support server, fort example over the Internet. This allows support data, such as calibration data for test strips, to be obtained over the network. Other types of support data may also be downloaded, such as drivers for new types of external devices, updated firmware, data analysis and tracking applications, and so forth. It will be appreciated, therefore, that the on-site of expandability through the use of external diagnostic devices connected through a universal data port and network support services implements a powerful and flexible paradigm for the future of home-based healthcare diagnostics. With this type of system, each user can customize his or her on-site device to provide a blood pressure monitor along with an array of test strip readers, other diagnostic devices, and data tracking and analysis applications, as selected to meet that individual's needs and p-references.

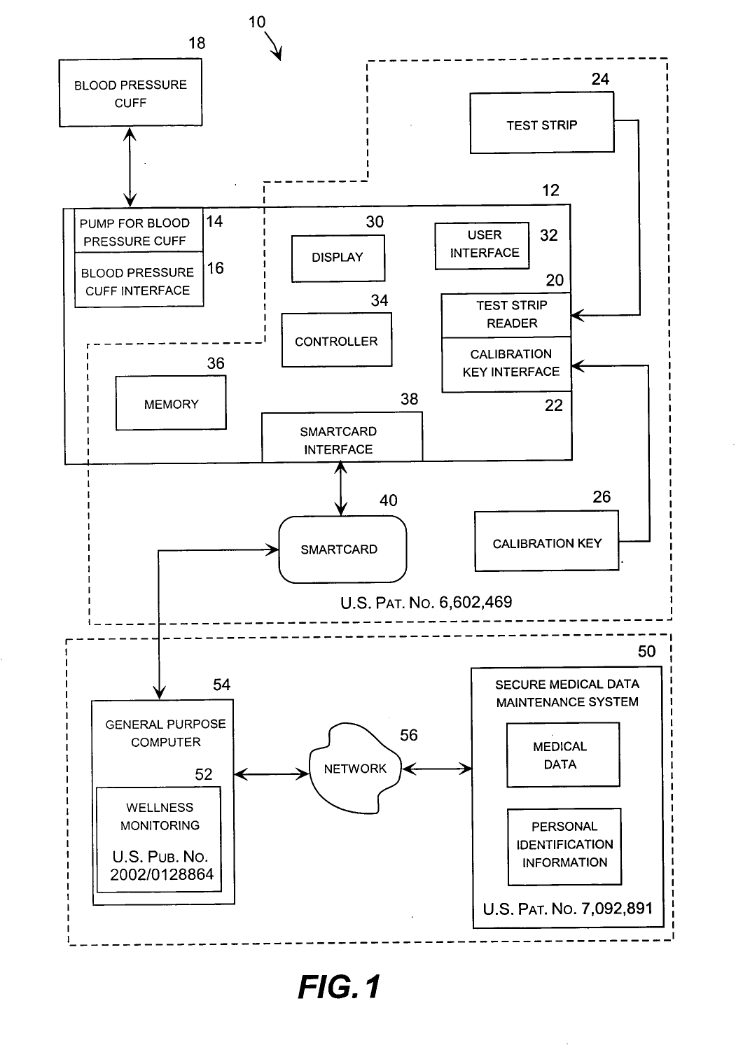

FIG. 1 is a functional block diagram of an on-site health diagnostic device 10 and related systems. This particular embodiment includes combination monitor 12 that includes a blood pressure monitor and a blood cholesterol monitor. The blood pressure monitor includes a pump 14 and a blood pressure cuff interface 16 that works in conjunction with a blood pressure cuff 18. The cholesterol monitor typically includes a test strip reader 20 for use with disposable test strips 22 and calibration key interface 24 for receiving a calibration key 26 for use with the rest strips. In particular embodiment, the cholesterol monitor may be as described in U.S. Pat. No. 6,602,469, which is incorporate herein by reference. In addition, the medical data created by the diagnostic device may be stored in a network-based secure medical data maintenance system 50 as described in U.S. Pat. No. 7,092,891, which is also incorporate herein by reference.

The medical data may also form part of a wellness monitoring system 52 that typically runs on a general purpose computer 54, which may interact with the secure medical data maintenance system 50 over a network 56 such as the Internet, as described as described in U.S. patent application Publication No. 2002/0128864, again incorporated herein by reference. The wellness monitoring system 52 allows the user to monitor a number of healthcare diagnostics measured by the on-site healthcare diagnostic device 10 along with other factors, such as diet and exercise. That is, the present invention includes the integration of a blood pressure monitor into the healthcare diagnostic devices described in these prior references, and contemplates the use of any or all of this technology in connection with the on-site device, as expanded to include the blood pressure monitor.

It should also be understood, however, that the on-site healthcare diagnostic device 10 may include a blood sugar monitor in addition to or instead of the blood cholesterol monitor, in combination with the blood pressure monitor. As noted above and described in greater detail below, the on-site monitor is expandable to work with any number of external diagnostic devices, and therefore is not limited to a single test strip reader. Nevertheless, it should also be appreciated that an on-site device that includes a blood pressure monitor in combination with a blood cholesterol monitor deployed as a special purpose device is a desirable combination. A on-site device that includes a blood pressure monitor in combination with a blood sugar monitor deployed as a special purpose device is also a desirable combination. Of course, either type of special purpose device can be external to include both a blood cholesterol monitor and a blood sugar monitor through the use of an external test strip reader connected to the device through a universal data port.

As shown in FIG. 1, the blood pressure monitor and test strip reader both use common features of the on-site device, including a display 30, a user interface 32, a controller 34, a memory 36, and data interface 38 that typically uses some type of removable memory storage device 40. For example, in a particular embodiment the display 30 may be a small liquid crystal display (LCD), the user interface 32 may be a three-button keypad, the data interface 38 may be a smartcard drive, and the controller 34 and memory 36 may be any of a variety of suitable alternatives.

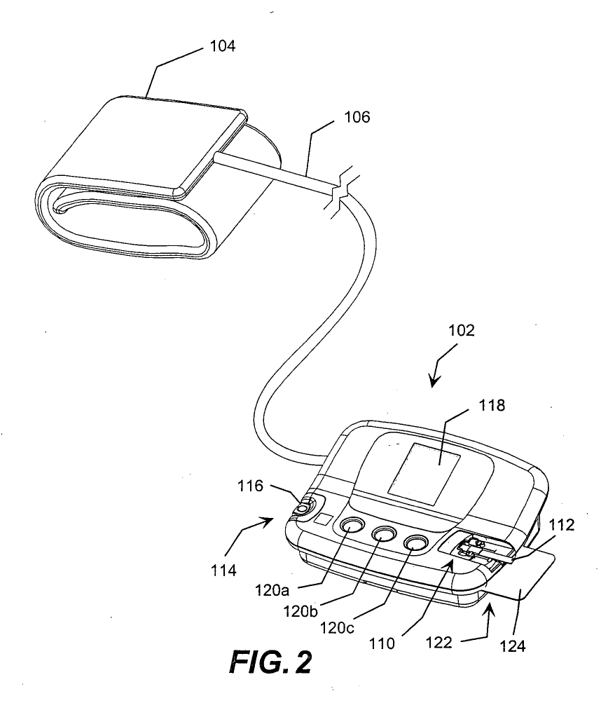

FIG. 2 is a perspective view of a particular embodiment of on-site health diagnostic device 100 that includes a blood pressure monitor and test strip reader, in this example an optical blood cholesterol test strip reader. This particular on-site device 100 includes a special purpose combination monitor 102 that connects to a blood pressure cuff 104 by way of a pneumatic and electric cable 106. The pump and control electronics for operating the blood pressure cuff 104 are contained within the combination monitor 102, which displays the test results and stores the resulting data. The operation of the blood pressure monitor is otherwise conventional and in accordance with well known blood pressure monitoring techniques.

The on-site monitor 102 also includes an optical test strip reader 110 shown with a test strip 122 inserted, and romkey socket 114c shown with a romkey 116 inserted. Generally described, each package of test strips come with a small romkey that contains calibration data for the associated test strips. The user inserts the romkey 116 in the socket 114, which configures the monitor with calibration data for the associated test strips, as represented by the test strip 112, inserted into the test strip reader 110 once an appropriate sample of blood has been applied to the test strip.

The on-site monitor 102 also includes a number of common components that support the blood pressure monitor and the cholesterol monitor. In addition to an internal controller and memory, the common components include a small LCD display 118, a three button keypad including buttons 120a-c, and a data drive 122. In this particular monitor, the data drive is a smartcard drive show with a smartcard 124 inserted into the drive. There operation of the device is intentionally simple to facilitate use without substantial training or study. Although the specific button operational protocol is a design choice, the left button 120a may be a “blood pressure button”; the center button 120b may be a “cholesterol button”; and the right button 120c may be a “memory button.”

For this particular example, to conduct a blood pressure test, the user puts on the blood pressure cuff 104 and depresses the blood pressure button 120a to initiate the test. Once the test is completed, the result is displayed on the display 118. To save the test result, the user presses the memory button 120c. Similarly, to conduct a cholesterol test, the user places a test strip with an appropriate blood sample in the test strip reader in the test strip reader 110 and depresses the cholesterol button 120b to initiate the test. Once the test is completed, the result is displayed on the display 118. Again, to save the test result, the user presses the memory button 120c. The user can also view saved test results by pressing the memory button 120c first, and then toggling through saved blood pressure test results using the blood pressure button 120a, or toggling through saved cholesterol test results using the cholesterol button 120b. Additional functionality for deleting test results, downloading test results to the smartcard 124, and other features are also accessed through the buttons and display. Operation of the device is intended to be straightforward and easily grasped from the configuration and labeling of the device.

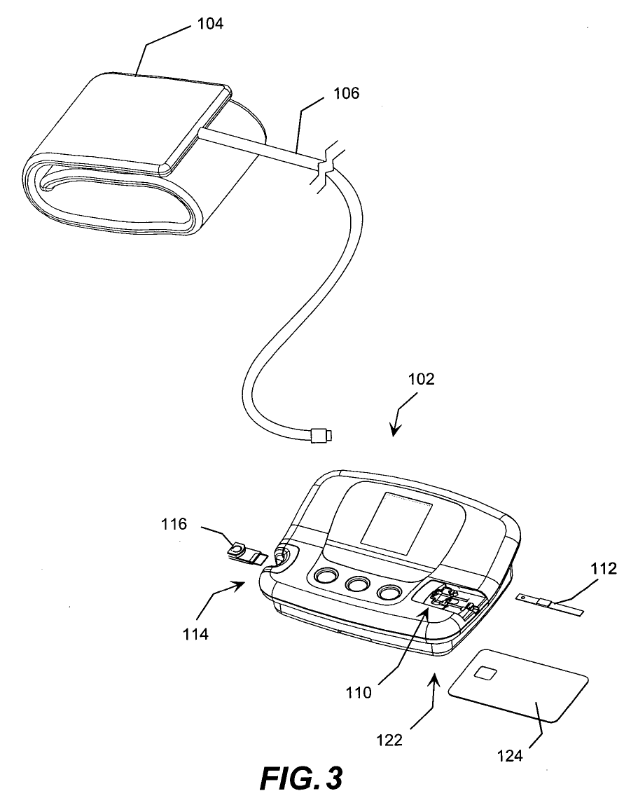

FIG. 3 is a partially exploded view of the on-site health diagnostic device 100 of FIG. 2 showing removable components in removed positions. Specifically, monitor 102 can be operated with a minimal number of parts that are designed to go together in an intuitively obvious way. Specifically, the blood pressure cuff cable 106 may be easily plugged into or removed from a blood pressure port on the monitor 102, the test strip 112 may be may easily inserted into or removed from removed from the test strip reader 110, the romkey 114 may be easily plugged into or removed from removed from the romkey socket 116 and the smartcard 124 may be easily inserted into or removed from removed from the smartcard drive 122. This makes for very easy operation and a minimal number of parts for the user to keep up with.

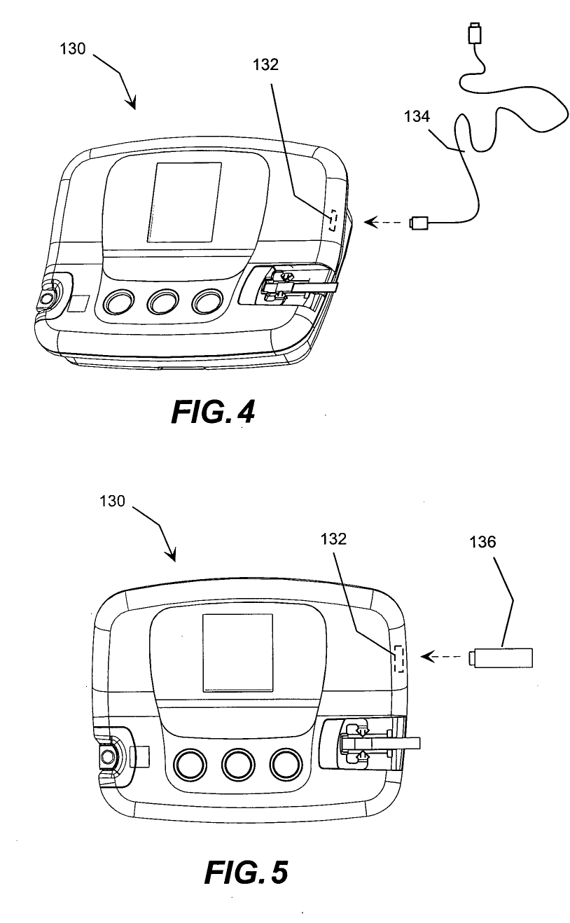

FIG. 4 is a perspective view of a slightly modified on-site health diagnostic device 130 that includes a universal data port, in this case a USB port 132 suitable for receiving a USB cable 134. FIG. 5 shows this embodiment with a USB memory stick 136. The USB port 132 may be included in addition to or as an alternative to the smartcard drive. The USB port 132 may also be used to connect the on-site device to a general purpose computer, a network, an external diagnostic device, a printer, or any other suitable resource. Including a universal data port provides the on-site device with expandability, flexibility and increased functionality by integrating the device with external sources including additional diagnostic devices, applications running on a general purpose computer, network-based support resources, and so forth.

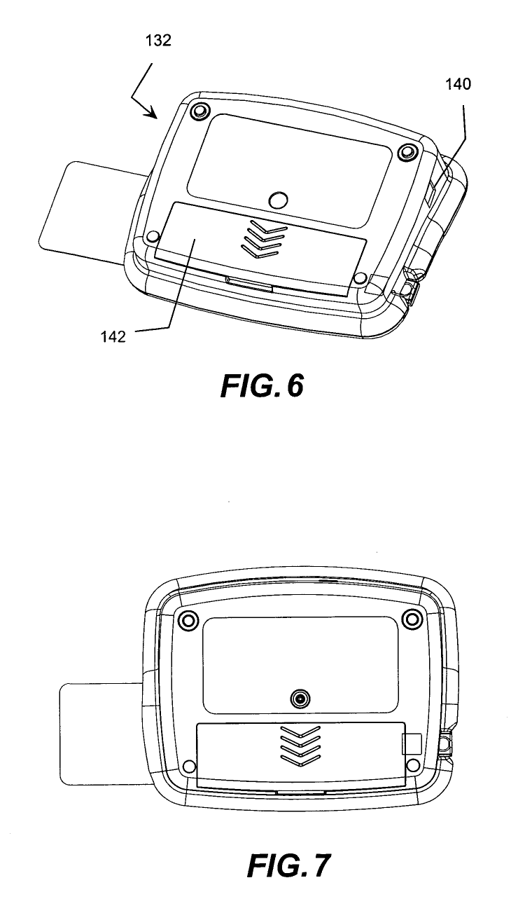

FIG. 6 is a perspective rear view and FIG. 7 is a rear view of the on-site health diagnostic device 130. These views show the blood pressure port 140 that receives the cable that goes to the blood pressure cuff. The USB port 132 may be located in a similar location on the opposing side of the device. The rear views also show a battery cover 142 and other features on the rear of the device.

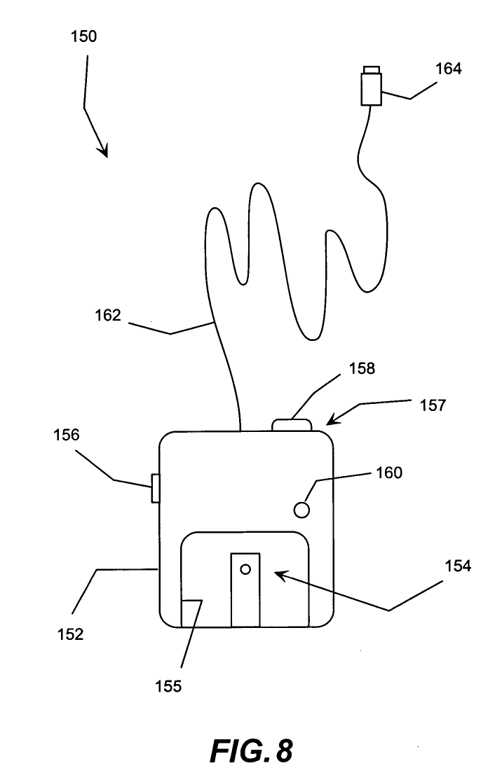

FIG. 8 is a front view of an external test strip reader 150 for use with the on-site health diagnostic device 130. The external test strip reader includes a small housing 152 about the size of a book or matches that includes an optical or biosensor test strip sensor 154, which is typically set within a an indentation 155 large enough to accommodate a person's thumb as they maneuver a test strip into the reader. The test strip reader also includes a “press to test” button 156 and an indicator light 158 that typically blinks while a test is in process and remains on until the test strip is removed to indicate a completed test. The test strip reader may also include a romkey socket for receiving a calibration key. However, the romkey socket may be omitted for applications in which the calibration key may be located elsewhere, for example on the host diagnostic device 130. As another alternative for application in which the external test strip reader is used in conjunction with a host diagnostic device or a general purpose computer, the calibration data may be obtained on demand from either the computer or a remote application server. This particular test strip reader also includes a cable 160 and USB connector 162 for connectivity. However, other types of connectivity may be utilized, such as wireless “blue tooth” or any other suitable type of data link.

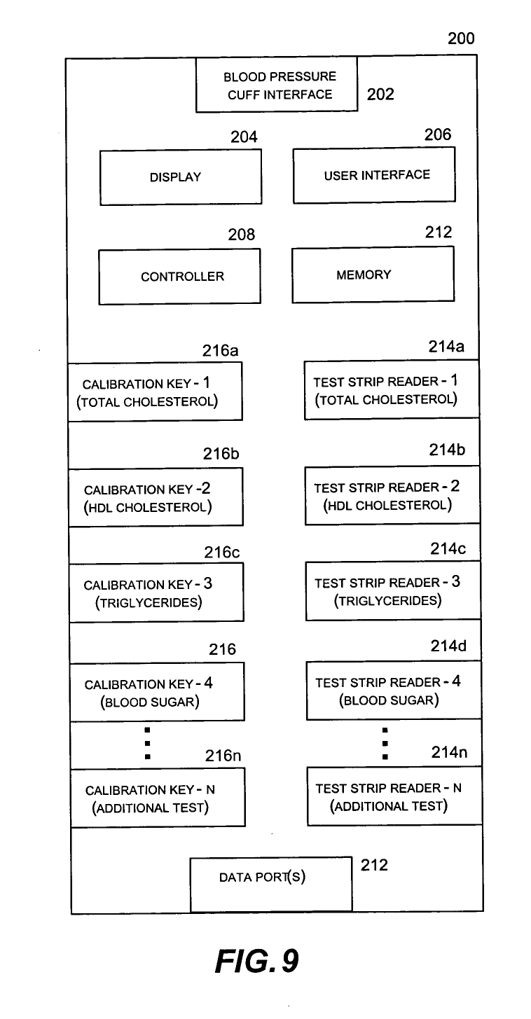

FIG. 9 is a block diagram of a on-site health diagnostic device 200 with a blood pressure cuff interface 202 and a multiple of test strip readers that all use common resources, including a display 204, user interface 206, controller 208, memory 210 and one or more data port(s) 212. The multiple strip readers 214a-n each have an associated calibration key 216a-n. The pump for the blood pressure cuff and one of the test strip readers are typically be located on-board the device, as shown in FIGS. 4 and 5, while additional test strip readers may be added as external test strip readers as shown in FIG. 8. In an alternative embodiment, the on-site health diagnostic device 200 may be general purpose computer with the pump for the blood pressure cuff and all of the test strip readers configured as external accessories to the general purpose computer. The use of the external test strip readers and other external devices gives the on-site health diagnostic device 200 virtually unlimited flexibility and expandability. This enables a wide range of important combinations to be supported by the manufacturer and selected by users at their own discretion.

For example, a first test strip reader 214a and associated calibration key 216a may be configured to conduct a total cholesterol test, a second test strip reader 214b and associated calibration key 216b may be configured to conduct a HDL cholesterol test, a third test strip reader 214c and associated calibration key 2106c may be configured to conduct a triglyceride test. With these diagnostic tests, the on-site health diagnostic device 200 may compute an LDL value and produce a full lipid panel measurement. The on-site healthcare diagnostic device may further a fourth test strip reader 214d configured to conduct a blood sugar test, and so forth. Any suitable algorithm for computing an LDL cholesterol value based on measurements of total cholesterol, HDL cholesterol and triglycerides may be used to produce the full lipid result from these measured values.

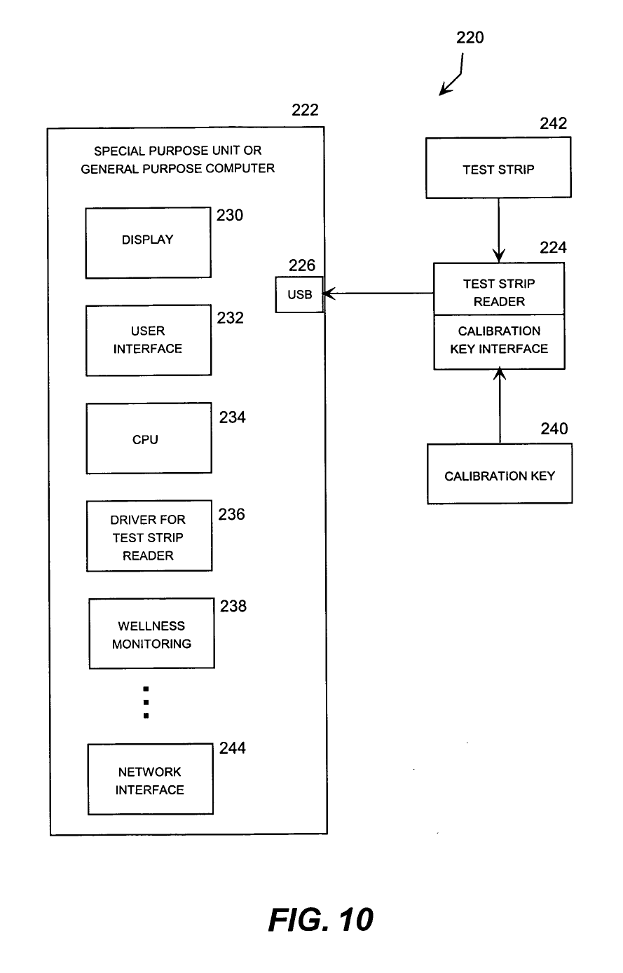

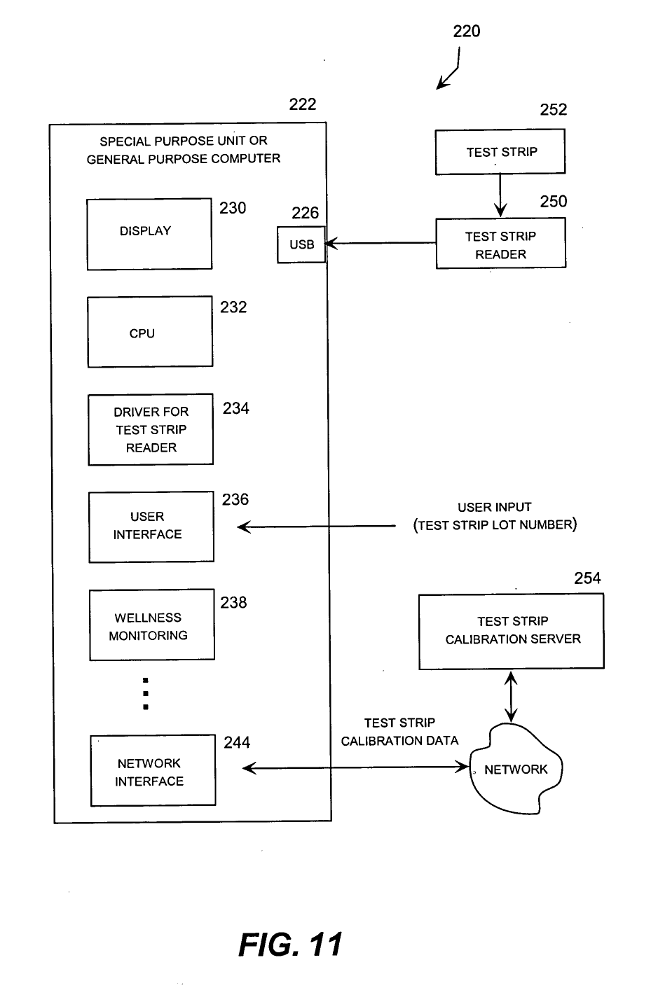

FIG. 10 is a block diagram of a on-site health diagnostic device 220 implemented with a general purpose computer 222 and an external test strip reader 224 connected to the computer through a general purpose data port, in this example a USB port 226. The general purpose computer 222 includes typical resources, such as a display 230, a user interface 232 (e.g., mouse and keyboard), and a central processing unit 234 operable for running applications to support the use of the computer as a healthcare diagnostic device. In particular, these applications may include a driver 236 for the test strip reader and software for storing and analyzing diagnostic test results, such as a wellness monitoring application 238. In this example, the test strip reader 224 includes a socket for receiving a calibration key 240 that contains calibration data for an associated set of test strips, as represented by the test strip 242. However, the calibration key 240 may be eliminated by using network interface 244 and network-based support, as shown in FIG. 11.

FIG. 11 is a block diagram of the on-site health diagnostic device 200 of FIG. 10 modified to use a test strip reader 250 for reading test strips, represented by the test strip 252, without the use of a calibration key. In this alternative, a user inputs a test strip lot number, which is typically printed on the test strips or their packaging. The on-site health diagnostic device 200 then uses the lot number to download the appropriate calibration data from a test strip calibration server 254. Also, calibration data tied to specific lots of test strips is typically used with optical test strips but may not be used with other types of test strip readers, such as biosensors. Calibration keys or network-supplied calibration data may not be required for some types of diagnostic devices. Nevertheless, network support is shown to illustrated illustrate this capability, which may be expanded beyond calibration data to any type of network-based support that may be appropriate for a particular diagnostic device.

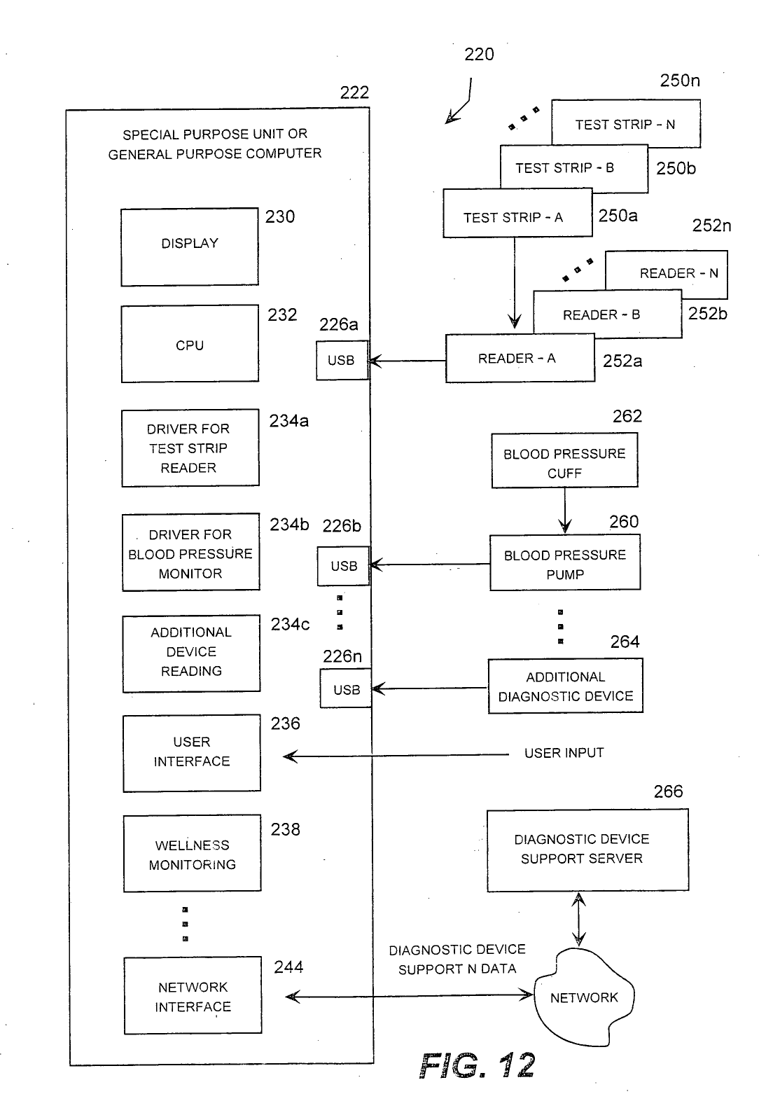

FIG. 12 is a block diagram of a generalized, expandable on-site health diagnostic device 200. In view of the features described above, it will be appreciated that the on-site health diagnostic device 200 can be generalized to work with any number of external test strip readers 250a-n and associated test strips 252a-n an external blood pressure pump 260 and cuff 262, and additional diagnostic devices, as represented by the additional diagnostic device 264. One ore more of these devices may utilize network based support from a diagnostic device support server 266, as needed. In this generalized embodiment, any particular diagnostic device, including the blood pressure pump, is optional and may be included or omitted on demand at the user's discretion. Accordingly, the invention may be practiced, for example, as a general purpose computer with a selectable set of external test strip reader accessories that can be connected to the computer through one or more universal data ports. Supporting software residing on the computer itself and/or accessible through network connectivity can be used to conduct diagnostic test using the external test strip reader individually or on-site, as described previously in connection with the full lipid panel test. It will be apparent to those skilled in the art that many changes and substitutions can be made to the embodiments of the invention herein described without departing from the spirit and scope of the invention as defined by the appended claims and their full scope of equivalents.

1. A on-site healthcare diagnostic device comprising a pump for a blood pressure monitor and a test strip reader deployed within a common housing, wherein the blood pressure monitor and test strip reader utilize common components of the device including a display, user interface, and controller.

2. The on-site healthcare diagnostic device of claim 1, wherein the test strip reader is configured to measure blood cholesterol content in a sample of blood applied to a test strip read inserted into the reader.

3. The on-site healthcare diagnostic device of claim 1, wherein the test strip reader is configured to measure blood sugar content in a sample of blood applied to a test strip read inserted into the reader.

4. The on-site healthcare diagnostic device of claim 1, further comprising an external test strip reader connectable to the common housing through a universal data port.

5. An on-site healthcare diagnostic device, comprising:
a general purpose computer comprising a data port;
one or more test strip readers connectable to the general purpose computer through the said data port; and
software running on the general purpose computer operable for driving said one or more test strip readers connected to the universal data port to operate in conjunction with a display, a user interface and a controller resident as part of the general purpose computer to function an as a healthcare diagnostic device.

6. An on-site healthcare diagnostic device as in claim 5 wherein said computer further includes a memory containing calibration data for said one or more test strip readers.

7. An on-site healthcare diagnostic device as in claim 5 wherein said computer includes a network interface for connecting to an off-site computer.

8. An on-site healthcare diagnostic device as in claim 7 and further including an off-site computer connected to said network interface, said off-site computer including a memory containing calibration data for said one or more test strip readers.

9. An on-site healthcare diagnostic device as in claim t wherein said one or more test strip readers are capable of reading a test strip for measuring an analyte selected from the group consisting of LDL cholesterol, HDL cholesterol, total cholesterol, triglycerides, glucose and creatinine

10. A method of measuring the concentration of an analyte in a whole blood sample, said method comprising:
connecting a dry test strip reader to a personal computer;
applying said whole blood sample to said dry test strip;
inserting said dry test strip into said test strip reader;
reading said dry test strip and producing data representative of said concentration of said analyte;
communicating said date to said personal computer; and
displaying on said personal computer said concentration of said analyte.

11. A method as in claim 10 and further comprising using calibration data stored on said personal computer to determine said concentration of said analyte.

12. A method as in claim 11 and further comprising sending said calibration data to said personal computer via a network.

13. A method as in claim 10 wherein said analyte is selected from the group consisting of LDL cholesterol, HDL cholesterol, total cholesterol, triglycerides, glucose and creatinine.
